(19) United States
(12) Patent Application Publication
(10) Pub. No.: US 2002/0064545 A1
(43) Pub. Date: May 30, 2002

(54) Formulations and methods for insect control

(76) Inventors: Stephen J. Mattingly, San Antonio, TX (US); David L. Johnson, San Antonio, TX (US)

Correspondence Address: ROBERT W. STROZIER, SUITE 930, 2925 BRIARPARK DRIVE, HOUSTON, TX, 77042, US

(21) Appl. No.: 09/951,833

(22) Filed: Sep. 11, 2001

Related U.S. Application Data
(63) Continuation of application No. 09/529,581, filed on Jun. 23, 2000, now abandoned.
Application No. 09/529,581 is a 371 of international application No. PCT/US98/21511, filed on Oct. 13, 1998.
(60) Provisional application No. 60/061,841, filed on Oct. 14, 1997.

(51) Int. Cl. A01N 25/08; A01N 25/00; A01N 63/00; A01N 65/00; C12N 1/22

(52) U.S. Cl. 424/410; 424/405; 424/84; 424/488; 424/93.1; 424/93.3; 435/252

(57) ABSTRACT

An oat bran-dried milk particulate mixture containing a bacterial pesticide such as one or more of a viable Gram negative organism, e.g. Pseudomonas, Enterobacter and Serratia marcescens has been found to eliminate or decrease populations of cockroaches and other insects. With an appropriate bait formulation containing bacteria, insects, including carpenter ants, termites and fire ants of any variety, can be controlled. Termite bait of course would preferably involve a cellulosic material. Carpenter ants may be baited more properly by a particulate mixture comprising, for example, peanut extract (peanut butter or the like). An important aspect of the present invention is including an appropriate bacterial pesticide with insect food. The pesticide is preferably slow-acting, not killing the insects immediately.

### BACKGROUND OF THE INVENTION

The red imported fire ant, Solenopsis invicta, was accidently imported from South America into the United States in the 1930s. During the intervening time period, it has spread throughout the southern states and Puerto Rico and is estimated to infest over 150 million hectares (Lofgren, 1986a). Its high population density correlates with man's activities, which has made it one of the most significant health and agricultural pest ant species (Adams, 1986). Its potent sting and injected alkaloid venom has resulted in a larger number of hypersensitivity reactions than seen with bee stings (Adams and Lofgren, 1981). Its effect on agricultural production of a variety of crops including soybeans, potatoes, corn, citrus, okra and others is well known (Vander Meer, 1986). The preferred diet of the adult red imported fire ant appears to be carbohydrate (Vander Meer et al., 1995). The bait used in the present microbial-based formulation contains >60% carbohydrate and is taken down into the mound min. after application. Attempts to control the red imported fire ants have included extensive use of several approaches: persistent chlorinated hydrocarbon insecticides (Adams, 1986); vegetable oil phagostimulant/active ingredient solvent (Banks et al., 1985); subterranean fogging devices (Amdro Fire Ant Insecticide, 1987); Amdro®, containing a chemical-based insecticide (Logic Fire Ant Bait, technical data, commercial brochure), and Logic®, containing fenoxycarb, a growth regulator, which when ingested by the queen prevents the development of eggs that would normally develop into worker ants (Logic Professional Fire Ant Bait, product label, Terminix International, Inc.). In Texas attempts are underway to used phorid flies in the biocontrol of imported fire ants (L. E. Gilbert Laboratory, U. T. Austin, 1997). As far as the inventors are aware, there are no microbial-based formulations to control the fire ant population. The present invention involves the use of several species of soil- and water-based bacteria in sufficiently large populations to colonize the digestive tract of the fire ant and cause death, in a manner yet to be determined, of all occupants of the mound. The use of a carbohydrate-based formulation encourages both ingestion of the bait, while also providing large surface areas for bacteria to attach during lyophilization.

The control of insect pests, particularly in environmentally safe manner is a perennial problem. For reasons of health and aesthetics, it is desirable to control insect populations. Imported red fire ants (Solenopsis invicta and other strains) have proven to be pests and potential health risks in many parts of this country. Biologically safe methods for control of fire ants and other insects have been less than completely satisfactory. The avermectin-producing Streptomyces avermitilis has been used to produce avermectin which was thought to be usable in the control of fire ant population. Other chemical insecticides have been used in attempts to suppress insect pests. The present invention comprises, in one embodiment, use of a special bait, most preferably with a mixture of microorganisms, for the control of fire ants and other insects. Various other suppression or eradication systems have been tested and yet insect control, as exemplified by the imported fire ant problem remains unsolved, despite the use of various insecticides.

### SUMMARY OF THE INVENTION

An oat bran-dried milk particulate mixture containing a bacterial pesticide such as one or more of a viable Gram negative organism, e.g. Pseudomonas, Enterobacter and Serratia marcescens has been found to eliminate or decrease populations of cockroaches and other insects. With an appropriate bait formulation containing bacteria, insects, including carpenter ants, termites and fire ants of any variety, can be controlled. Termite bait of course would preferably involve a cellulosic material. Carpenter ants may be baited more properly by a particulate mixture comprising, for example, peanut extract (peanut butter or the like). An important aspect of the present invention is including an appropriate bacterial pesticide with insect food. The pesticide is preferably slow-acting, not killing the insects immediately.

In one important embodiment, the present invention most generally involves a method for effectively administering population-controlling materials to a fire ant colony. The method involves preparing a dried particulate mixture comprising a carbohydrate and at least one preferably viable fire ant population-controlling microbe as a bacterial pesticide. The mixture is then applied in proximity to a fire ant mound or made available in the area patrolled by the insect. In one preferred embodiment, the carbohydrate is included in a cereal bran. One effective cereal bran is oat bran. The carbohydrate may also include dried milk and to a residue of a thioglycollate bacterial broth. A preferable bacterial pesticide is at least one of a Pseudomonas aeruginosa, Enterobacter agglomerans, Enterobacter aerogenes, and Serratia marcescens. Other insect pathogens or toxins such as that of Bacillus thuringiensis, for example, may be utilized for certain insects. B. thuringiensis is a Gram positive soil bacteria that has been found to produce insecticidal toxins called Bt toxins. The purified or transgenically expressed Bt toxins are commonly used as insecticides in agriculture. Therefore, the present invention may further comprise other bacterial insecticides for insects, particularly fire ants, or other materials having negative effects on insect populations.

Another aspect of the present invention is a dry particulate mixture comprising carbohydrate and at least one bacterial insecticide deleterious to fire ant populations. The bacteria is most preferably viable and, in an important embodiment, at least one of Pseudomonas aeruginosa, Enterobacter agglomerans, Enterobacter aerogenes , and Serratia marcescens. A cereal bran containing carbohydrate milk solid and thioglycollate medium solids have been found particularly beneficial in both attracting insect consumption and stabilizing viable bacteria.

In one embodiment, the present invention concerns the effective application of isolated strains of Pseudomonas aeruginosa, Enterobacter agglomerans, Enterobacter aerogenes and Serratia marcescens to fire ant colonies. The bacterial strains are preferably contained on carbohydrate-rich particles that are retrieved and ingested by the ants. Subsequent to an effective application, fire ant colonies were found to become abandoned and contain only dead ants.

The present invention broadly involves a method for controlling insect populations. This method comprises preparing or obtaining a particulate mixture comprising a carbohydrate and a possible insect population depletor, such as a bacterial insecticide. An effective amount of the mixture is applied to an area populated by insects. The insects consume the material and/or retrieve it for consumption by other insects. A preferred particulate mixture comprises a cereal bran. In a preferred method the agent insecticide or pathogen is a bacterium. In another preferred method the agent is at least one of Pseudomonas aeruginosa, Enterobacter aerogenes, Enterobacter agglomer ans, and Serratia marcescens most preferably isolated from commerical grease traps. These methods appear to be effective for many insects, including cockroaches, carpenter ants, fire ants, and termites.

The particulate material should include an appropriate “bait” to induce consumption by target insects. For example, the carbohydrate in oat bran and/or dried-milk thioglycollate bacterial broth residue with is an effective “bait” for fire ants and cockroaches. It is envisioned that cellulose would attract termites and that other insects would be attracted by the same or other dietary components or flavorings.

### DESCRIPTION OF ILLUSTRATIVE EMBODIMENTS

The present invention involves finding a biologically safe and effective method to control undesired insect populations and the spread of pests such as imported red fire ants. Generally the insect bait of the present invention may be prepared as follows:bacterial pesticides, including Pseudomonas aeruginosa, Enterobacter aerogenes, Enterobacter agglomerans and Serratia marcescens, are grown individually in bacterial media to log phase or, preferably, stationary phase. The bacteria are removed from the bacterial media by centrifugation, filtration or any other means known to those of skill in the art. The bacteria are then resuspended in a medium comprising skim milk and/or other appropriate suspension media such as e.g. thioglycollate medium. Dry oat bran or some other attractive food source for insects is then added to the bacterial suspension. The materials are mixed thoroughly and then lyophilized or otherwise dried without significantly damaging bacteria viability. Following lyophilization or drying, the dry material is weighed and is ready for use. It is believed that the bacteria used in the methods described are entirely safe to humans and animals. Preferably, for treatment of fire ant mounds, a quantity of bait containing at least 5×10⁹ to 1×10¹³ bacteria is applied to each mound. In this invention, mixtures of different bacterial strains or a single bacterial strain may be ultimately combined with a carbohydrate-rich bait in a dry state for broadcast in the area or application to fire ant mounds.

Preliminary results affirm that the microorginisms to be included in the bait should be viable, although essentially dry.

The following examples are included to demonstrate preferred embodiments of the invention. It should be appreciated by those of skill in the art that the techniques disclosed in the examples which follow represent techniques discovered by the inventors to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention. For example, numerous carbohydrate-rich particulate baits may be used so long as they are regarded as food and retrieved by ants to the colony for ingestion. It is believed that any Gram negative bacteria, particularly of the genera described herein are useable as bacterial insecticides.

Also, other microbial agents may also be substituted for the particular bacteria exemplified. Pseudomonas aeruginosa, Enterobacter agglomerans, Enterobacter aerogenes, and Serratia marcescens are all Gram negative bacteria. Gram negative bacteria are divided into several classifications. E. agglomerans and S. marcescens are both members of the family Enterobacteriaceae. P. aeruginosa is a member of the family Pseudomonadaceae. Other types of bacteria, including other Gram negative or even Gram positive bacteria that are bacterial insecticides, may be used in the present invention. Although the bacterial strains used in the illustrative embodiments were generally obtained from water effluent of grease traps it is beleived that a laboratory strain of Pseudomonas aeruginosa or other organisms obtained from another source also demonstrate insecticidal activity against fire ants and the other insects. Therefore effective bacterial strains are not limited to those obtained from grease traps.

### EXAMPLE 1

Six strains of bacteria (one of Pseudomonas aeruginosa, one of Enterobacter aerogenes one of Enterobacter agglomerans, and three of Serratia marcescens) were isolated from the effluent of a commercial grease trap and are available from BioStim, L.L.C. These strains were grown individually in 1 liter quantities of Trypticase Soy Broth (Difco) for 18-24 hours, with a final yield of 10⁹-10¹² bacteria/ml. The bacteria were removed from the Trypticase Soy Broth by centrifugation at 10,000×g for 20-30 minutes. The bacteria were then resuspended in 100 ml of 10% skim milk (Difco) and, when mixing the strains, all six bacterial strains were mixed together in equal proportions. Dry Quaker Oat Bran was added at 453 g/l liter of original Trypticase Soy Broth medium. The material was mixed to cookie dough consistency and lyophilized until dry. Following lyophilization, the dry material was weighed and ready for use. Approximately 2.5-5.0 g were applied to each fire ant mound in the subsequent Examples unless stated otherwise. As little as 1 g was found to often have similar results.

### EXAMPLE 2

A field test was conducted by David L. Johnson on fire ant mounds located on property next to the office of BioStim L.L.C., San Antonio, Tex. in May 1997. The results were 100% kill of the mounds within two weeks (including queens and larva). The 2nd tests were conducted in Mr. Johnson's back yard during the month of June, 1997 at his residence in San Antonio. Mr. Johnson applied the formulation to 10 separate fire ant mounds and there was 100% kill within two weeks. Mr. Johnson conducted a 3rd round of tests in July 1997 in which he baited 3 of five mounds in his front yard in close proximity to two other satellite mounds. The 3rd set of tests resulted in a complete kill in two weeks of the three original mounds and a corresponding kill of the satellite mounds in week three. Dr. Stephen J. Mattingly conducted tests on his personal property on seven mounds in August, 1997 and reported all mounds free of living fire ants in two weeks. A fourth set of tests was conducted on seven mounds located on residential property in August, 1997. All mounds were found to be free of live fire ants within two weeks.

### EXAMPLE 3

The oat bran-dried milk-Pseudomonas, Enterobacter, Serratia-containing particulate mixture of the present invention has been noted as eliminating or decreasing cockroach populations. With appropriate bait or flavoring, target insects may include, among others, carpenter ants, cockroaches, termites and fire ants of any variety. Termite bait of course would preferably involve a cellulosic material. Carpenter ants may be baited more properly by a particulate mixture comprising, for example, substances such as peanut extract (peanut butter or the like). An important aspect of the present invention is including an appropriate pathogen. The pathogen is preferably slow-acting, not killing the insects immediately. It is believed that, because of fire ants' preference for dietary carbohydrate, a variety of toxins or bacteria pathogenic to fire ants may be included in a particulate mixture and applied to fire ant mounds effectively. When the fire ants retrieve the carbohydrate-rich particles to the colony for ingestion, the toxin or pathogen may have effects in the insect population. Choices of carbohydrate-rich material attractive to fire ants are widely available. Particular toxins or pathogens may be readily tested by the techniques described herein and effective materials identified. Those of skill in the art will understand how the basic successful fire ant control technique may be established. Of great importance is a particulate form attractive to fire ants for consumption. This is most likely to be a material rich in carbohydrates. In addition, the pathogen or toxin should not be readily detectable by the ants and preferably is slow-acting.

All of the compositions and methods disclosed and claimed herein can be made and executed without undue experimentation in light of the present disclosure. While the compositions and methods of this invention have been described in terms of preferred embodiments, it will be apparent to those of skill in the art that variations may be applied to the compositions and methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### EXAMPLE 4

Further experiments to determine the insecticidal activity of the insect bait of the present invention were conducted using ants colonies with 8-12 in. mounds and enclosed in one gallon glass containers. The ambient conditions were uniformly dry with temperatures ranging from 60-75° F. Approximate number of ants, and fire ant activity when slightly disturbed, were observed upon application of the samples, and weekly thereafter. The results are shown in Table 1. Treatment Packets 1 through 6 contained individual bacterial strains while Packet 7 contained a combination of all six strains in one packet. Packet 1 contained a first isolated strain of Serratia marcescens; packet 2 the isolated strain of Enterobacter agglomerans; packet 3 a second isolated strain of Serratia marcescens; packet 4, an isolated strain of Pseudomonas aeruginosa; packet 5, an isolated strain of Enterobacter aerogenes; packet 6, a third isolated strain of Serratia marcescens. The packets were prepared by a procedure similar to that described above. Packet 7 is a formula currently marketed by BioStim, L.L.C. for treatment of grease traps and drain lines. Packets containing 5 gram quantities were found to be effective for killing ants while 1 gram packets were found to be ineffective indicating that the efficacy of the bait may be dose dependent. When applied in the 5 gram quantities, all formulations resulted in complete kill of the tested ant colony within 2 to 4 weeks. When no fire ant activity was observed upon disturbance of the mound, the soil was checked for live ants and discarded if none were found. This result is indicated in Table 1 by the description “all dead” for approximate number of ants observed.

TABLE 1
Approximate number of Fire Ants/Activity Level

| | | Week | Week | Week | Week | Week | Week | Week |
|---|---|---|---|---|---|---|---|---|
| Treatment | Initial | 1 | 2 | 3 | 4 | 5 | 6 | 7 |
| Control | >100 | >50 | >50 | >50 | >50 | >50 | >50 | >50 |
| (untreated) | high | mod | mod | mod | mod | mod | mod | mod |
| Packet 7 | >100 | >100 | >100 | >100 | all | | | |
| | | | | | dead | | | |
| (5 gram) | high | low | low | low | none | | | |
| Packet 6 | >100 | <20 | <20 | all dead | | | | |
| (5 gram) | high | low | none | none | | | | |
| Packet 4 | >100 | >50 | >50 | all dead | | | | |
| (5 gram) | high | low | low | none | | | | |
| Packet 2 | >100 | >50 | all dead | | | | | |
| (5 gram) | high | low | none | | | | | |
| Packet 5 | >100 | >50 | <20 | all dead | | | | |
| (5 gram) | high | mod | mod | none | | | | |
| Packet 3 | >100 | >50 | <20 | all dead | | | | |
| (5 gram) | high | low | ND | none | | | | |
| Packet 1 | >100 | >50 | few | all dead | | | | |
| (5 gram) | high | mod | low | none | | | | |
| Packet 4 | >100 | >100 | >50 | >50 | | | | |
| (1 gram) | high | high | mod | high | | | | |
| Packet 7 | >100 | >100 | >50 | >100 | | | | |
| (1 gram) | high | high | mod | high | | | | |

### Example 5

Preliminary Report on Initial Field Trial of BioStim Microbial Fire Ant Bait

### Materials and Methods

The test site was a non-bearing pecan orchard located in eastern Burelson County in the Brazos River flood plain. Fire ants were most likely of the multiple-queen type at a density averaging nearly 430 mounds per acre. Trees within the orchard were on a 45-foot spacing. Plots consisted of an area bounded by three inter-tree spaces, i.e., 135 feet, on a side, or 0.42 acres. The central 45×45 foot square was used as the sample area to allow an ample treated buffer. All active fire ant mounds within the sample area were counted. A 45 foot buffer was left untreated around all sides of all plots.

Initial mound controls were made on the morning of Jul. 24, 1998. A mound was considered active if a dozen or so ants rushed to the surface upon light disturbance with a pointed tool handle. The mounds counts were arrayed from highest to lowest and divided into four equal groups (replications). Treatments were assigned within replications so that the total number of active mounds for each treatment were as equal as possible. Treatments included:

The BioStim bait was prepared as follows: Six Strains are grown separately in 3 liters each of Tryptic Soy Broth (Difco) overnight at 35C. The cells are separated by centrifugation and suspended in 1 liter of 10% skim milk. The mixture is then added along with 1 liter of Thioglycollate Broth without indicator (Difco) to 1500 grams of dry Quaker Oat Bran and mixed to cookie dough consistency. The material is then lyophilized to dryness, mixed to break up clumps in a food mixer, and packaged for use. Application was made after 5:30 p.m. on Jul. 27, 1998. Evaluations were made on Aug. 3, 10, 17 and 25, 1998 by counting all active mounds within each plot's sample area, as described above. The treatment/rate and method are shown in Table 2.

TABLE 2

| Treatment | Rate | Method |
|---|---|---|
| 1) untreated control | — | — |
| 2) Logic ® (1% fenoxycarb) | 1.5 lbs/acre | broadcast |
| 3) BioStim Bait | 1.5 lbs./acre | broadcast |
| 4) BioStim Bait | 4.5 lbs./acre (equiv.) | placed in center of |
| | | sample area |

Results

Foraging ants were very attracted to the BioStim product and began carrying it off almost as it hit the ground. It appeared to somewhat more attractive than Logic, though both baits were readily retrieved by the ants. Results of post-treatment counts are shown in Table 3:

TABLE 3
Mean number of active mounds per plot (4 replications)

| Treatment | pre-count | 1 wk | 2 wk | 3 wk | 4 wk |
|---|---|---|---|---|---|
| Untreated | 20.25 a | 19.75 a | 23.75 a | 18.50 a | 17.50 a |
| Logic | 20.25 a | 20.00 a | 16.75 a | 12.50 a | 16.00 a |
| BioStim, | 20.00 a | 16.50 ab | 14.50 b | 11.75 a | 19.50 a |
| broadcast | | | | | |
| BioStim, | 20.00 a | 13.00 b | 13.00 b | 9.00 a | 18.00 a |
| central | | | | | |
| F | 46.03 | 10.72 | 11.86 | 1.70 | 3.20 |
| | (replct) | | | | |
| P | 0.0001 | 0.0012 | 0.0008 | 0.2270 | 0.0573 |
| R² | 0.9684 | 0.8773 | 0.8877 | 0.5315 | 0.6811 |

Means in the same column followed by different letters are significantly different (P<0.05) using PC SAS ANOVA procedures. Means separated using Duncan's multiple range test.

The BioStim product resulted in a fairly rapid reduction in active mound numbers that also appears to be rate-related. Activity then appears to have leveled off. Active mound numbers are significantly lower (P<0.05) for the centrally-placed BioStim product versus untreated and Logic plots at one wk post-treatment. All treatments are significantly lower than untreated plots at two wk, but all treatments are statistically similar after that point.

Weather during the test was extremely hot and, initially, extremely dry. Two significant rain events occurred during the evaluation period that likely caused untreated-plot mound numbers to fluctuate. The fast initial drop in colony numbers in the central-placement plots indicates a potential rate response. In which case future tests should included treatments in a range of five to 20 pounds per acre, for instance. The product is extremely attractive to ants, regardless of its efficacy.

### REFERENCES

The references in the following list are incorporated in pertinent part by reference herein for the reasons cited in the text.

Adams, “Agricultural and medical impact of the imported fire ants,” In: Fire ants and leaf-cutting ants: Biology and management, C. S. Lofgren and R. K. Vander Meer (eds.), Westview Press, Boulder, Colo., p. 48-57, 1986.

Adams and Lofgren, “Red imported fire ants [Hymenoptera: Formicidae]: Frequency of sting attacks on residents of Sumter County, Ga.,” J. Med. Entomol., 18:378-382, 1981.

Amdro Fire Ant Insecticide, Produce label, American Cyanamide Co., Wayne, N.J., 1987.

Banks, Lofgren, Williams, “Development of toxic baits for control of imported fire ants,” In: Pesticide formulations and application systems: 4th Symp., Special Tech. Publ. 875, Amer. Soc. Test. Mater., Philadelphia, pp. 133-143, 1985.

L. E. Gilbert Laboratory, U. T. Austin, “Using phorid flies in the biocontrol of imported fire ants in Texas,” http://uts.cc.utexas.edu/gilbert/research/fireants/fireant.html, 1997.

Lofgren, “The economic importance and control of imported fire ants in the United States,” In: Economic impact and control of social insects, S. B. Vinson (ed.), Praeger, N.Y., p. 227-256, 1986a.

Logic Fire Ant Bait, Technical Data, Commercial brochure.

Logic Professional Fire Ant Bait, Product Label, Terminix International Inc.

Vander Meer, “The trial pheromone complex of Solenopsis invita and Solenopsis richteri,” In: Fire ants and leaf-cutting ants, Biology and management, C. S. Lofgren and R. K. Vander Meer (eds.), Westview Press, Boulder, Colo., p. 201-210, 1986.

Vander Meer et al., “Fire ant phagostimulants,” Florida Entomologist, 78(1):145-154, 1995.

1. A dry particulate mixture for insect population control, the mixture comprising:
carbohydrate-containing vegetable matter ingested by said insects; and
at least one bacteria pesticidal to said insects.

2. The particulate mixture of claim 1 further defined as comprising protein.

3. The particulate mixture of claim 1 wherein the bacteria pesticidal to insects is at least one of Pseudomonas aeruginosa, Enterobacter agglomerans, Enterobacter aerogenes, and Serratia marcescens.

4. The mixture of claim 1 wherein the bacteria are Gram negative.

5. The mixture of claim 1 where the bacteria are isolated from commerical grease trap eluent.

6. The mixture of claim 1 further comprising dry milk solids.

7. The mixture of claim 1 further comprising dried thioglycollate bacterial medium.

8. A method for administering a bacterial pesticide to a fire ant colony, the method comprising:
preparing a dried particulate mixture comprising a carbohydrate and a bacterial pesticide; and
applying an effective amount of said mixture in proximity to a fire ant mound.

9. A method for administering a bacterial pesticide to a fire ant colony, the method comprising:
preparing a dried particulate mixture comprising a carbohydrate and-viable bacteria; and
applying an effective amount of said mixture in proximity to a fire ant mound.

10. A method for administering a bacterial pesticide to a fire ant colony, the method comprising:
preparing a dried particulate mixture comprising a carbohydrate and viable bacteria isolated from a commerical grease trap; and
applying an effective amount of said mixture in proximity to a fire ant mound.

11. A method for administering a bacterial pesticide to a fire ant colony, the method comprising:
preparing a dried particulate mixture comprising a carbohydrate and Gram negative bacteria; and
applying an effective amount of said mixture in proximity to a fire ant mound.

12. A method for administering a bacterial pesticide to a fire ant colony, the method comprising:
preparing a dried particulate mixture comprising a carbohydrate and at least one viable bacteria isolated from a commerical grease trap;and
applying an effective amount of said mixture in proximity to a fire ant mound.

13. The method of claim 8 where the particulate mixture further comprises a cereal bran.

14. The method of claim 8 where the particulate mixture further comprises oat bran.

15. The method of claim 8 where the mixture comprises dried milk.

16. The method of claim 8 where the bacterial pesticide is Gram negative.

17. The method of claim 8 where the bacterial pesticide is at least one of Pseudomonas aeruginosa, Enterobacter agglomerans, Enterobacter aerogenes and Serratia marcescens.

18. A method for the control of imported fire ant populations, the method comprising
obtaining isolated strains of Pseudomonas aeruginosa, Enterobacter
agglomerans, Enterobacter aerogenes and Serratia marcescens effective
in the inhibition of fire ant colony growth and maintenance;
preparing a mixture of at least one of said bacterial strains; and
applying said mixture to fire ant colonies in a manner inducing fire ants to retrieve and consume said strains.

19. A dry particulate mixture for fire ant control comprising carbohydrate and a bacteria deleterious to fire ant populations.

20. The mixture of claim 19 where the bacteria is at least one of Pseudomonas aeruginosa, Enterobacter agglomerans, Enterobacter aerogenes and Serratia marcescens.

21. A method for controlling insect populations, the method comprising:
preparing a dry particulate mixture comprising a carbohydrate and a bacterial pesticide;
applying an effective amount of said mixture to an area populated by said insects; wherein said insects consume or retrieve said mixture for the consumption of other insects.

22. The method of claim 21 where the particulate mixture comprises a cereal bran.

23. The method of claim 21 where the particulate mixture comprises oat bran.

24. The method of claim 21 wherein the carbohydrate is included in milk solids.

25. The method of claim 21 where the mixture further comprises a residul of a thioglycollate baterial medium.

26. The method of claim 21 wherein the bacterial pesticide is at least one of Pseudomonas aeruginosa, Enterobacter agglomerans, Enterobacter aerogenes, and Serratia marcescens.

27. The method of claim 21 wherein the insects are cockroaches or imported fire ants.

28. The method of claim 21 where the insects are termites and the carbohydrate is cellulose.

29. The method of claim 21 wherein the insects are carpenter ants.
